(19) United States
(12) Patent Application Publication
(10) Pub. No.: US 2025/0295519 A1
(43) Pub. Date: Sep. 25, 2025

(54) Genital Protective Device with Enhanced STD Prevention

(71) Applicant: Raffi Khajak Marashlian, Los Angeles, CA (US)

(72) Inventor: Raffi Khajak Marashlian, Los Angeles, CA (US)

(21) Appl. No.: 18/614,829

(22) Filed: Mar. 25, 2024

(51) Int. Cl. A61F 6/04

(52) U.S. Cl. CPC A61F 6/04; A61F 2006/047

(57) ABSTRACT

The present disclosure relates to a protective device designed for the prevention of sexually transmitted diseases. The device includes a first portion made of a flexible material, forming a hollow tubular cavity with a closed end for enclosing the penis and an open end equipped with a first elastic ring. This ring is designed to snugly fit around the base of an erect penis. Extending from the first elastic ring, a second portion of the device is also made from a flexible material and is designed to form a larger cavity than the first. This second cavity, equipped with a second elastic ring, is intended to encircle and securely cover the base of the testicles and the penis shaft, thus providing comprehensive coverage of the male genitals.

### FIELD OF INVENTION

The present invention relates generally to the field of sexual health devices, specifically to an improved protective device designed to enhance the prevention of sexually transmitted diseases (STDs) by offering comprehensive coverage of the male genitalia, including both the penis and the testicles.

### BACKGROUND

The advent and widespread use of condoms have marked a significant milestone in the realm of sexual health, primarily by offering a barrier method to prevent unwanted pregnancies and reduce the transmission of sexually transmitted diseases (STDs). Despite their efficacy, conventional condoms possess inherent limitations that compromise their protective capability. Specifically, the design of standard condoms, which typically covers only the shaft of the penis, leaves significant areas of the male genitalia, such as the testicles, exposed to potential contact with infected skin or fluids. This exposure presents a notable vulnerability, as it disregards the transmission risks associated with skin-to-skin contact, which can facilitate the spread of STDs like genital warts, herpes, and other pathogens capable of thriving on the external genitalia.

Moreover, the conventional method of applying and securing condoms does not address the need for comprehensive protection, focusing solely on the shaft of the penis. This oversight in design and application fails to mitigate the risk of infection through contact with the exposed areas not covered by the condom. The conventional approach to condom application, which involves rolling the condom down to the base of the penis shaft, inherently neglects the anatomical proximity of the testicles to potential sources of infection, thereby leaving these areas vulnerable during sexual activity.

Recognizing these critical limitations, there has been a compelling necessity to innovate beyond the traditional condom design to encompass a more holistic approach to STD prevention. The traditional condom's inability to offer full coverage and its resultant failure to safeguard against the transmission of infections via exposed skin areas have underscored the urgent need for a protective device that can offer an enhanced barrier against STDs. This need has catalyzed the development of an advanced protective device designed to extend the protective coverage to include the testicles, thereby addressing the significant gap in protection and offering a more comprehensive solution to safer sexual practices. The inception of this invention reflects a thoughtful response to the limitations and drawbacks inherent in current condom designs, aiming to significantly reduce the risk of STD transmission through an innovative approach that encapsulates the entire genital area. This development signifies a pivotal shift towards achieving higher safety standards in sexual health protection.

It is within this context that the present invention is provided.

### SUMMARY

The invention introduces a novel protective device aimed at significantly enhancing the prevention of sexually transmitted diseases (STDs) by addressing the limitations inherent in conventional condoms. By incorporating a dual-portion design, it extends protective coverage beyond the penis shaft to include the testicles, an area left vulnerable in traditional condom designs. This device features a hollow tubular cavity for the penis and a larger, second cavity with elastic bands on both openings to ensure a secure fit around both the penis and the testicles. By doing so, it mitigates the risk of STD transmission through skin-to-skin contact with exposed areas not covered by standard condoms, providing a more comprehensive solution to safer sexual activity.

In some embodiments, the flexible material of the first and second portions is selected from latex, polyurethane, polyisoprene, and nitrile. This choice allows for a range of materials that can accommodate various user sensitivities and preferences, ensuring that the device can be widely used by a diverse population.

In some embodiments, the second cavity is approximately spherical in shape, which allows it to conform closely to the natural shape of the testicles. This feature is designed to improve comfort and ensure that the device remains securely in place, providing effective coverage without causing undue pressure or discomfort.

In some embodiments, the device further comprises a lubricant applied to the interior surfaces of the first and second portions. This lubrication facilitates the application and removal of the device, enhancing user experience by reducing friction and potential discomfort.

In some embodiments, the first and second elastic rings are integrated with an anti-slip texture. This texture is designed to enhance the securement of the device during use, preventing slippage and ensuring that the device stays in place even during vigorous activity.

In some embodiments, the first tubular portion and the second wider portion are manufactured as a single, continuous piece. This design simplifies the manufacturing process and eliminates seams or joints that could potentially weaken the structure or cause discomfort during use.

In some embodiments, the device is designed to be reversible, allowing for easier removal by inverting the second portion over the first portion. This reversibility feature simplifies the removal process, making the device more user-friendly and reducing the risk of spillage or contamination during removal.

In some embodiments, the second portion has a smaller thickness than the first portion.

In some embodiments, the second portion is formed of a different, softer material than the first portion.

Overall, the invention provides a protective device with several features aimed at improving the prevention of sexually transmitted diseases by ensuring comprehensive coverage, enhancing comfort and securement, and simplifying application and removal.

### BRIEF DESCRIPTION OF THE DRAWINGS

Various embodiments of the invention are disclosed in the following detailed description and accompanying drawings.

FIG. 1 illustrates an isometric side view of the protective device in a fully extended state.

FIG. 2 illustrates an isometric front view of the protective device in a folded state as it would appear when removed from the packaging.

FIG. 3A illustrates an isometric side view of the protective device during the initial stage of application over the penis shaft and testicles.

FIG. 3B illustrates the continuation of the application process with the protective device being drawn over the testicles.

FIG. 3C illustrates the protective device fully applied, with both the penis shaft and testicles securely enclosed.

FIG. 4 illustrates the initial stage of removing the protective device by inverting the second portion towards the tip.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention.

As used herein, the term “and/or” includes any combinations of one or more of the associated listed items.

As used herein, the singular forms “a,” “an,” and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The term “flexible material” as used herein refers to any material capable of conforming to the shape of an erect penis and the testicles, providing comfort and secure fit without compromising the integrity of the device. Example materials include latex, polyurethane, polyisoprene, and nitrile. These materials are selected for their elasticity, durability, and hypoallergenic properties, ensuring they can be used safely by individuals with varying sensitivities.

The “elastic ring” denotes a stretchable band designed to ensure the secure positioning of the protective device on the user's body. These rings are integrated at the open ends of both the first and second portions of the device, made from materials such as silicone or natural rubber, which offer flexibility and strength to maintain a snug fit around the penis base and the testicles. The elastic ring's design includes features such as anti-slip textures to prevent displacement during use.

### DESCRIPTION OF DRAWINGS

The present invention relates to a protective device designed to prevent sexually transmitted diseases (STDs) through enhanced coverage and securement features. At its core, the invention comprises two primary components: a first portion and a second portion, both crafted from a flexible material that ensures both comfort and effectiveness. The first portion is shaped as a hollow tubular cavity with a closed end to encase the penis, while the second portion extends from this to form a larger cavity designed to encompass the testicles. Integral to both portions are elastic rings positioned at their open ends to secure the device in place, preventing slippage and ensuring comprehensive protection during use.

The invention is distinguished by its approach to extend protective coverage beyond the penis to include the testicles, addressing a significant gap in the capability of existing protective devices. This extended coverage is crucial for reducing the risk of STD transmission in areas that standard condoms typically do not protect. By incorporating flexible materials such as latex, polyurethane, polyisoprene, or nitrile, the device can accommodate various user preferences and sensitivities, ensuring a broad applicability.

Furthermore, the inclusion of elastic rings at both the base of the penis and around the testicles provides a dual securing mechanism. This design not only enhances the protective capabilities of the device but also improves its stability and comfort during use. The application of lubricants to the interior surfaces of both portions of the device facilitates ease of use, enhancing the overall user experience by reducing friction and potential discomfort.

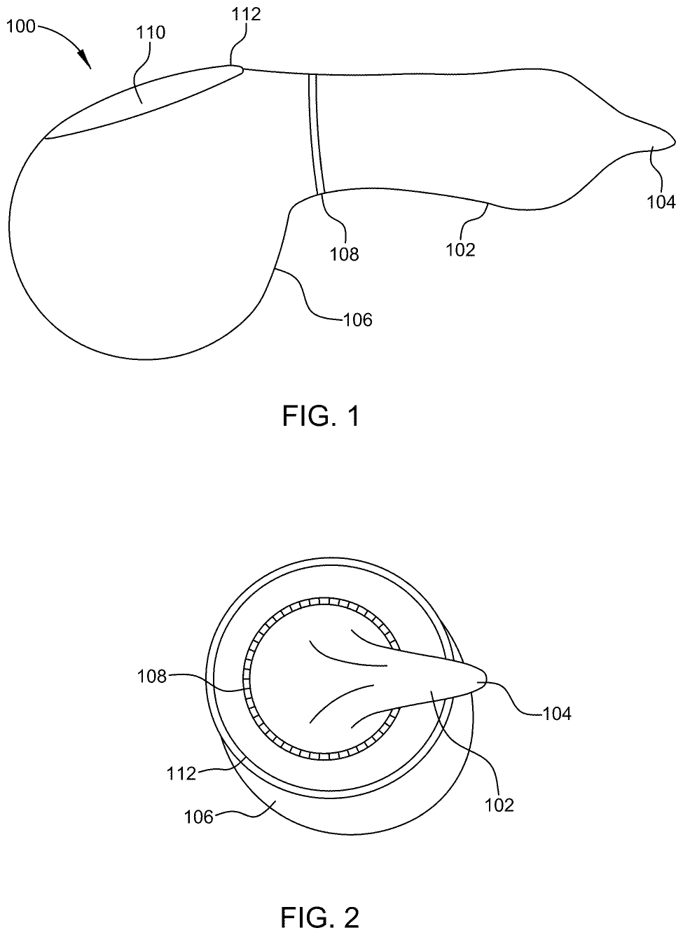

FIG. 1 depicts an isometric side view of the protective device, denoted as 100, showcased in its fully extended configuration for clarity. This view emphasizes the seamless integration of the tubular portion 102, terminating in a tip 104 at its closed end, indicative of the end designed to cover the penis. The device transitions at approximately its midpoint into a second, broader portion 106 via a first elastic ring 108. This transition is crucial for accommodating the testicles. An opening 110, positioned at the zenith of the second portion 106 and diametrically opposed to the tubular cavity, is encircled by a second elastic ring 112, ensuring a secure and snug fit around the base of the testicles and penis shaft.

FIG. 2 presents an isometric front view of the protective device 100 in a compact, folded state, illustrating how the device would appear when initially removed from its packaging. This representation provides an insight into the device's compactness and the ease with which it can be prepared for use.

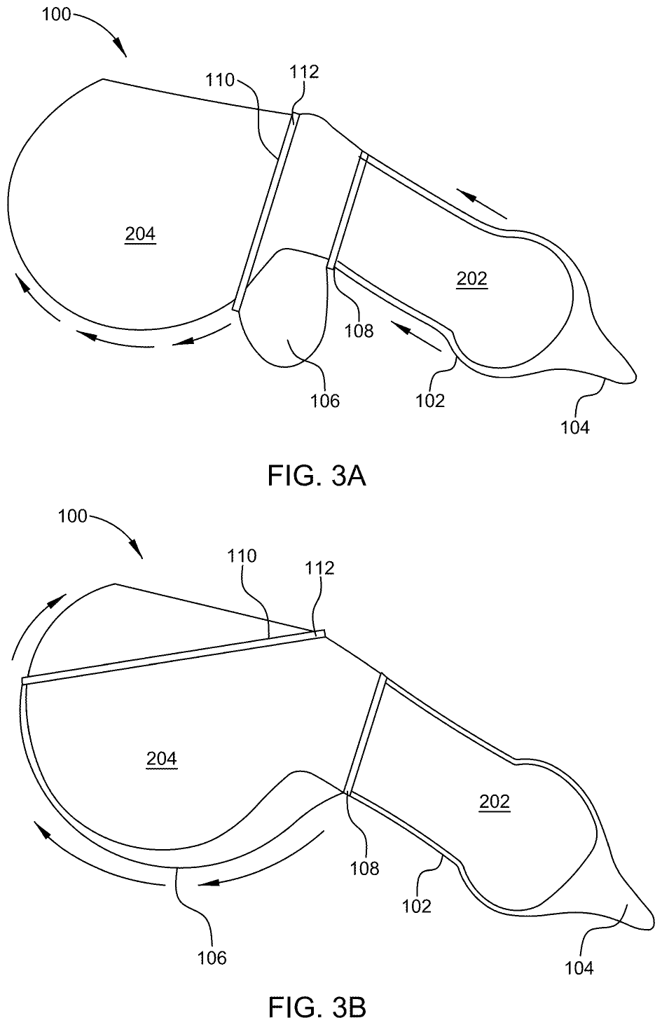

FIG. 3A offers an isometric side view detailing the initial phase of the protective device 100 being positioned over a penis shaft 202 and testicles 204. At this stage, the tubular portion 102 is shown drawn over the penis shaft 202, positioning the penis tip near the closed end tip 104, while the second, wider portion 106 is positioned in readiness to encase the testicles.

FIG. 3B captures the subsequent application phase, where the second portion 106 is in the process of being drawn over the testicles 204, highlighting the adaptability of the device to conform to the user's anatomy.

FIG. 3C illustrates the completion of the application process, showcasing the second portion 106 as it fully encloses the testicles. The second elastic ring 112 is depicted as fully encompassing the base of the penis shaft and testicles, illustrating the device's secure and comprehensive protective capability.

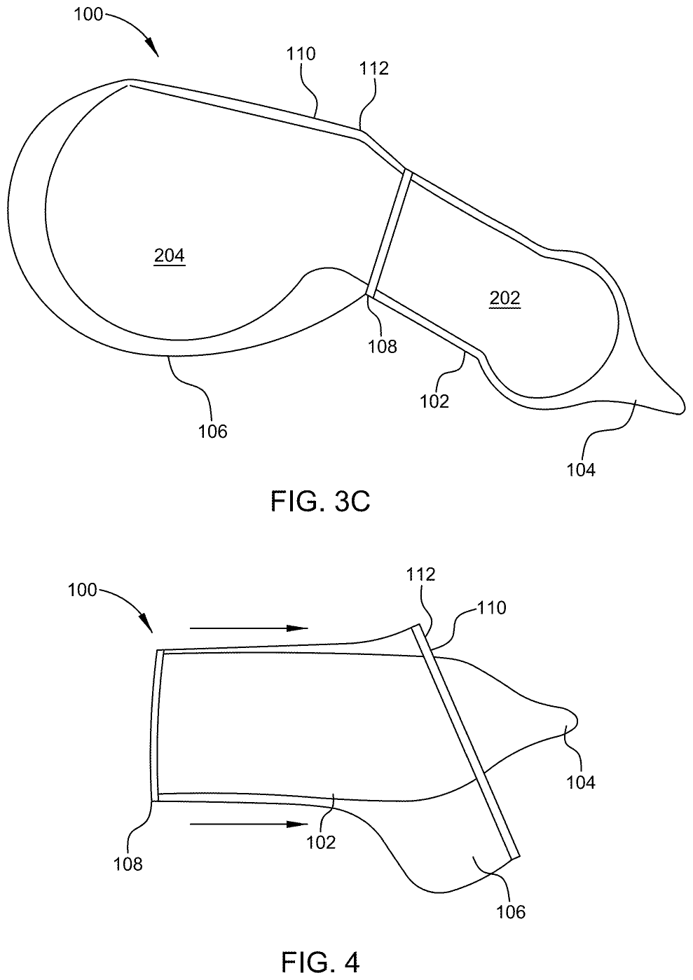

FIG. 4 reveals the initial stage of removing the protective device 100, where the second portion 106 has been inverted towards the tip, causing a portion of the tubular portion 102 to overlap itself. This step demonstrates the beginning of the removal process, emphasizing the ease with which the device can be taken off.

It is noted that, for increased comfort and due to the different forces likely to be experienced by the two portions, the second portion for covering the testicles may have a lower thickness and may be formed of as softer material than the first portion.

The protective device of claim 1, wherein the second portion is formed of a different, softer material than the first portion.

### CONCLUSION

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the protective device of the invention have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A system operable as a collaborative idea marketplace configured with a memory and one or more processors, the system comprising:
a user database, said user database comprising at least two categories of anonymous users including one or more owners and one or more collaborators; and
an idea database, said idea database including a dynamic database schema describing attributes of an idea owned by an owner of a plurality of owners in said user database,
the dynamic database schema includes attributes including one or more of an idea titles, an image describing or illustrating the idea, an idea description, and user interface elements for permitting user actions on said idea.

2. The system of claim 1, wherein the one or more collaborators of said user database, upon reviewing attributes of a particular idea, are presented with at least two action options.

3. The system of claim 2, wherein the action option includes one or more of buying or bidding said particular idea or offering to collaborate with an owner of said particular idea.

4. The system of claim 2, wherein the action options are succeeded by dynamic business rules.

5. The system of claim 4, wherein the dynamic business rules define financial terms and/or financial responsibilities, and legal terms or responsibilities.

6. The system of claim 1, wherein the owners are one or more of inventors, designers and/or makers.

7. The system of claim 1, wherein the idea description includes any form of multimedia content.

8. The system of claim 1, wherein the collaborators are permitted to select from the one or more idea titles to review idea information of the one or more ideas.

9. The system of claim 1, wherein the collaborators are permitted to submit one or more of a collaboration offer or a purchase offer for the one or more idea titles.

10. The system of claim 1, further comprising:
sending one or more collaboration offers or one or more purchase offers to the owners,
selecting, by an owner, a preferred offer from the one or more collaboration offers or one or more purchase offers from the one or more collaborators; and
adding or co-assigning a collaborator to a particular idea.

11. The system of claim 1, further comprising:
selecting by, a collaborator of the one or more collaborators, one or more idea titles;
filling out a particular offer, by the collaborator, comprising one or more of a collaboration offer or a purchase offer for the one or more idea titles; and
submitting the particular offer.
